(19) United States
(12) Patent Application Publication
(10) Pub. No.: US 2009/0306777 A1
(43) Pub. Date: Dec. 10, 2009

(54) APPARATUS FOR FIXING A LIGAMENT

(75) Inventors: Karl-Heinz Widmer, Merzhausen (DE); Jurg Bachler, Holstein (CH); Martin Bachler, Holstein (CH); Albert Moser, Niederdorf (CH); Roland Schaffner, Liestal (CH); Rudolf Koch, Oberdorf (CH)

Correspondence Address: GIBSON & DERNIER L.L.P., 900 ROUTE 9 NORTH, SUITE 504, WOODBRIDGE, NJ, 07095, US

(73) Assignee: BACHLER FEINTECH AG, Holstein (CH)

(21) Appl. No.: 11/909,185

(22) PCT Filed: Mar. 22, 2006
(86) PCT No.: PCT/IB2006/050881

§ 371 Date: Aug. 4, 2009

(30) Foreign Application Priority Data
Mar. 31, 2005 (CH) 00570/05

(51) Int. Cl. A61F 2/08; A61B 17/08; A61B 17/58

(52) U.S. Cl. 623/13.14; 606/151; 606/104

(57) ABSTRACT

The invention relates to an apparatus for fixing a ligament, which comprises a bone-anchoring element having a proximal end and a distal end which has a bore running in the longitudinal direction, and which comprises a clamping sleeve having a proximal end and a distal end, which clamping sleeve can be accommodated in the bore. The clamping sleeve is radially expandable, with the result that the ligament can be fixed between the clamping sleeve and the bore of the bone-anchoring element.

### BACKGROUND

The present invention relates to an apparatus for fixing a ligament in surgery and a method for the surgical repair of a ligament. It furthermore relates to a tool for use with the apparatus according to the invention.

Ligaments are extendable, fibrous connective tissue strands which flexibly connect the moveable parts of the skeleton but limit mobility to a functionally reasonable degree and stabilize it with regard to non-physiological movements. If ligaments are extended beyond their natural length, stretching of ligaments and tearing of ligaments may occur. In particular, tearing of the cruciate ligament in the knee joint is very frequent. The customary method for replacing the torn cruciate ligament is the use of a ligament transplant, in particular of an autologous transplant from the patella tendon or the semitendinosus tendon. In a known technique, the implant is taken from the tendon in such a way that a small bone block from the patella and the tibial bone remain at each of the ends. The transplant is then anchored in the knee joint so that the bone blocks can intergrow in each case tibially and femorally with the bone. After the transplant has been drawn in, it is fixed to the bone. Below, the terms fixed or fixation/fixing always mean the connection between two interfaces with high tensile and shear strength. What is important is that the implant is drawn in and fixed so that it is under a certain tension which is required for functioning of the joint but on the other hand the tension at each joint position is not so great that mobility is limited.

For drawing in, the ligament transplant is usually connected at both ends to filaments which are then led through bone canals so that the ligament is drawn in with the aid of the filaments. There are various approaches for fixing the ligament. Firstly, it is usual to fix the ligament itself and secondly it is also known that the ligament can be fixed by a looped-on filament. This means that, instead of fixing the ligament itself, the filaments connected to the ligament are fixed. The latter is customary, for example, in the fixing of cruciate ligament transplants which carry bone blocks at their ends.

For direct fixing, it is known, for example, that the ligament can be clamped or can be fixed with the aid of screws. Fixing filaments can be fixed by knotting to screws or other retaining apparatuses. Finally, many types of auxiliary apparatuses are known, by means of which the ligaments and/or retaining filaments are fixed, cf. for example WO98/33455, U.S. Pat. No. 6,099,568 and U.S. Pat. No. 6,336,940 B1. Furthermore, so-called interference screws are known for fixing a ligament and/or filament. U.S. Pat. No. 6,660,023 B2 discloses a screw where the filament is likewise fixed by a so-called interference fit. However, the screw has a channel in which the filament is guided. In the channel, the filament can be drawn in the longitudinal direction by application of a comparatively strong force. Nevertheless, compression is sufficiently strong to press the filament against the anchor element. Finally, U.S. Pat. No. 6,517,579 B1 discloses an anchor apparatus by means of which a ligament can be directly fixed. The apparatus has a plurality of parts. The ligament is led between a sleeve and the drilled channel in the bone. An element to be introduced into the interior of the sleeve in the manner of a screw presses the sleeve with the ligament against the drilled channel, with the result that the ligament is fixed. Additional fixing is furthermore achieved by a clamp-like covering which presses the ligament into the bone in the manner of a drawing pin.

US 2004/037094 describes a bone anchoring element which is provided with eyes and is screwed into the bone by means of a screwdriver-like tool. The length of the filament drawn through the eyes has to be adjusted in a relatively complicated procedure with the aid of an adjustable, clasp-like slide. US 2004/0098050 discloses an approximately can-shaped element which can be anchored in the bone and in which the adjustable length of the associated filament can be fixed by locking of the lid and associated transformation of an elastic material contained in the can.

However, the apparatuses known in the prior art have the disadvantage that the ligament is very frequently punctured for fixing, with the result that it may tear. Furthermore, it is generally not possible to adjust the tension of the ligament exactly since, as a result of fixing with the known aids and apparatuses, a change in length very frequently occurs. The theoretical requirement to be implemented in practice is that the fixing of the ligament must take place so that neither the ligament is further tensioned by the fixing nor the tension of the ligament declines. This is not ensured in the case of most known apparatuses. Thus, the ligament length is automatically changed on screwing in an interference screw. The same applies if the filaments are knotted under tension and fixed.

### SUMMARY

It is therefore an object of the invention to provide an apparatus for fixing a ligament which makes it possible to fix the ligament without uncontrolled effects on the length and hence the tension of the ligament. Furthermore, damage to the ligament during fixing is to be avoided.

The apparatus according to the invention for fixing a ligament has a bone-anchoring element and a clamping sleeve. The bone-anchoring element has a proximal end, i.e. an end located closer to the patient's body and a distal end, i.e. an end remote from the body of the patient, and has a bore running in the longitudinal direction. The clamping sleeve has a proximal and a distal end. It can be accommodated in the bore and is radially expandable. As a result of this radial expansion of the clamping sleeve, the ligament can be fixed between the clamping sleeve and the bore of the bone-anchoring element.

The method according to the invention for the surgical repair of a ligament comprises the following steps, after a replacement for the ligament to be repaired had been drawn in via at least one drilled channel: a) introduction of a bone-anchoring element (1) into the drilled channel and anchoring therein, the ligament or at least a filament (50) connected to it for fixing being led in a bore (4) of the bone-anchoring element, b) radial expansion of a clamping sleeve (10) accommodated in the bore, with the result that the ligament can be fixed between the clamping sleeve and the bore of the bone-anchoring element.

As is known, the ligament is drawn into the joint by means of holes drilled in the bone. The bone-anchoring element is introduced into this bone bore, the ligament or the filament or the filaments which is/are connected to the ligament being accommodated in the bore of the bone-anchoring element and being led through. The clamping sleeve is now introduced into the bore, this being designed so that the ligament or filament has sufficient play. This means that there is a sufficient distance between the clamping sleeve and the bone-anchoring element so that the filament or filaments or the ligament can be moved between these two elements. The necessary tension is now applied to the ligament, for example with the aid of a spring balance, and the ligament or the filaments are fixed. For this purpose, the clamping sleeve is radially expanded so that the ligament or the filament is clamped between clamping sleeve and bone-anchoring element and fixed thereby. With the aid of the apparatus according to the invention, both the ligament itself and at least one filament connected to the ligament can be fixed. The number of filaments is not decisive, so that the expressions “a filament” and “a plurality of filaments” can be used interchangeably below.

In particular, a spindle which is connected to the clamping sleeve is provided for expanding the clamping sleeve. The spindle in turn has a proximal and a distal end. It is led through the clamping sleeve. The diameter of the proximal end is greater than the internal diameter of the clamping sleeve. The radial expansion of the clamping sleeve is effected by drawing the spindle out of the clamping sleeve, with the result that the clamping sleeve is extended owing to the size difference of the proximal end of the spindle and of the internal diameter of the clamping sleeve. An advantage of this design is that the expansion of the clamping sleeve takes place from the proximal end to the distal end. This means that the filament or the ligament is first fixed in the vicinity of the body and only thereafter is the further part of the filament or of the ligament subsequently fixed by the continuing expansion. This has the advantage that the tension is fixed with the first fixing at the first point of the bone-anchoring element. It can no longer change as a result of the further fixing of the ligament or of the filament between clamping sleeve and bone-anchoring element.

According to an alternative embodiment, however, the radial expansion can also be effected by means of grub screws or similar elements. The introduction of a rivet would also be an alternative embodiment.

The bone-anchoring element is preferably designed in the manner of a bone screw with a hollow bore. It is preferably produced from metal with suitable roughening (e.g. coarsely blasted) but can also be produced from bovine or human bone material which has been appropriately pretreated for implantation. The roughening can be effected, for example, by coarse corundum blasting. What is advantageous about the roughening of the surface on the bone side is that the secondary fixation, i.e. the bone growth, is improved. The bone-anchoring element can also be produced from resorbable material, such as polylactide or the same type of resorbable materials as used for the so-called resorbable interference screws. It is introduced into the bone channel by screwing in. A screwdriver of our own design which is preferably cannulated so that the tension filaments can be led through it is provided for screwing in. The filaments serve as a guide during introduction of the bone-anchoring element into the drilled channel. This is extremely advantageous since the position of the bone-anchoring element is predetermined by the exit point of the drawn-through fixation filaments out of the bone bore. The bone-anchoring element can thereby also be mounted outside the body on the screwdriver and only thereafter be guided through the soft tissue channel to the drilled channel by the filaments in an accurate manner.

The clamping sleeve is preferably profiled, in particular ribbed, knurled or dimpled, on the outside, with the result that the ligament is particularly well fixed. The profiling may be transverse, oblique, rhombus-shaped, helical or in a comparable pattern with periodical change of depths and elevations. The outside of the clamping sleeve thus readily engages the filament or the ligament without damaging it. The clamping sleeve is preferably made of metal.

It is intended that the clamping sleeve and/or the bone-anchoring element be produced from a shape memory alloy which is also known by the name “memory metal”. This has the advantage that, for example, the radial expansion of the clamping sleeve takes place simply by virtue of the fact that it expands owing to the body temperature which is higher in comparison with room temperature, which the clamping sleeve assumes before it is introduced into the body. A manual expansion process, for example by means of a spindle, would thus be superfluous.

For the withdrawal of the spindle from the clamping sleeve and hence the radial expansion thereof, pliers of a particular design are provided. They are similar to pop rivet pliers. They moreover enable the filaments to be tensioned with the aid of additionally mounted tensioning devices with a defined, optionally also measurable tensile force and hence the ligament transplant to be pressed into the bone.

After insertion of the bone-anchoring element with the special screwdriver, the guide sleeve of the screwdriver remains on the top of the bone-anchoring element and thus forms a fixed working channel through the so-called soft tissue covering. The spindle of the pop rivet pliers is then introduced with the mounted clamping sleeve (10) through the working channel and, after expansion of the clamping sleeve is complete, the filaments are cut off using a customary filament cutter.

Preferred developments of the invention are shown in the figures, and the list of reference numerals is part of the disclosure. The invention is explained in more detail figuratively and by way of example with reference to these figures.

The figures are described in relation to one another and as a whole. Identical reference numerals denote identical components, and reference numerals with different indices indicate functionally identical or similar components.

### BRIEF DESCRIPTION OF THE DRAWING

FIG. 1 shows a section through an apparatus according to the invention in combination with a corresponding tool;

FIG. 2 shows the section corresponding to FIG. 1, the apparatus being shown in use according to the invention (with filament);

FIG. 3 shows two working examples of a bone-anchoring element according to the invention; each in perspective view (A, C) and in sectional view (B, D);

FIG. 4 shows a section through a clamping screw according to the invention;

FIG. 5 shows a side view of a spindle according to the invention; and

FIG. 6 shows a screwdriver according to the invention in partly cut-away side view (A, C with guide sleeve) and in section (B without guide sleeve).

### DETAILED DESCRIPTION OF THE EMBODIMENTS OF THE INVENTION

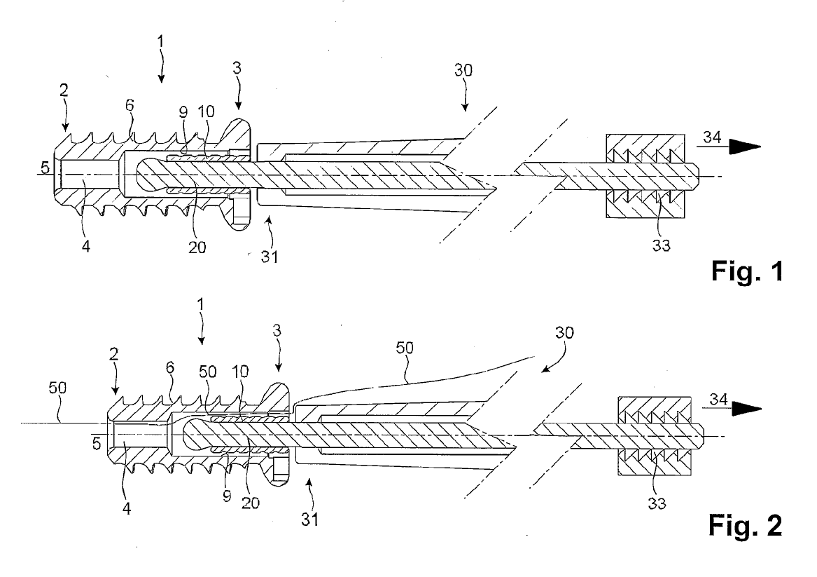

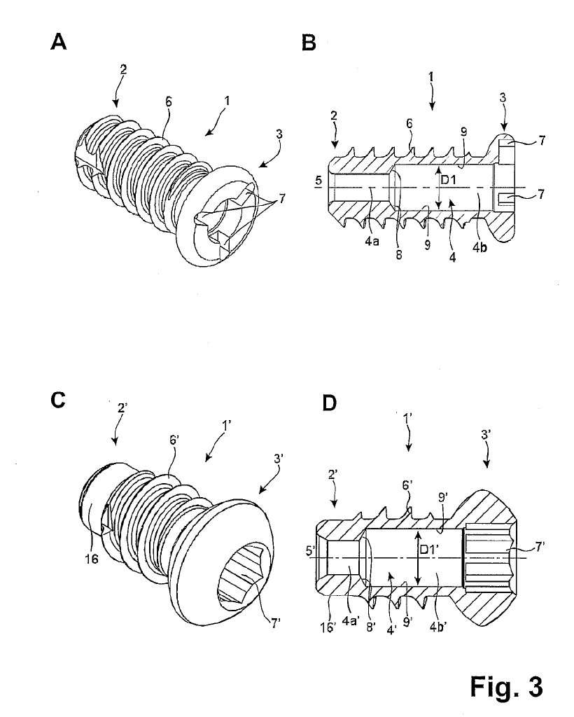

FIGS. 1 and 2 each show an apparatus according to the invention in section, as assembled ready for use, a filament 50 connected to a ligament (not shown) additionally being incorporated in FIG. 2. The apparatus according to the invention for fixing a ligament has a bone-anchoring element 1 which has a proximal end 2 and a distal end 3. The bone-anchoring element 1 has a bore 4 which runs along the longitudinal axis 5 of the bone-anchoring element or of the entire arrangement. As is evident from the diagrams of FIG. 3, the bone-anchoring element 1 has, on its outside, a thread, e.g. a bone thread, for example in sawtooth form. An engagement opening 7 through which the bone-anchoring element 1 can be engaged by means of a screwdriver or a similar tool and can be turned is furthermore provided. Consequently, the bone-anchoring element 1 can be introduced in a self-tapping manner into the drilled bone. The bore 4 preferably has two regions, a region 4a and a region 4b, the region 4b having a larger diameter compared with the region 4a. This results in an axial stop 8.

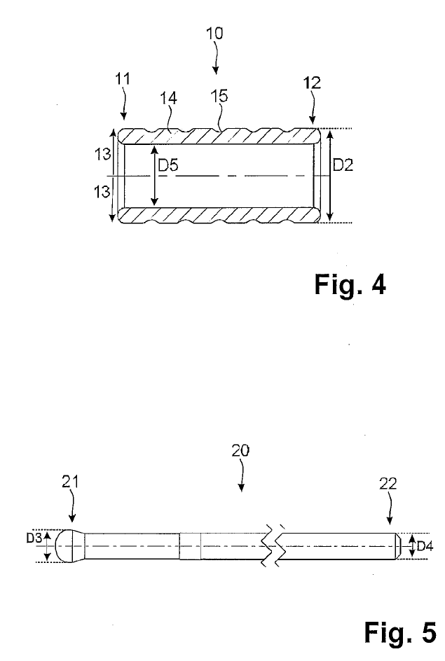

As is evident from FIGS. 1 and 2, a clamping sleeve 10 is introduced into the interior of the bone-anchoring element 1. The clamping sleeve 10 likewise has a proximal end 11 and a distal end 12 (FIG. 4) The clamping sleeve 10 is a hollow cylinder. The external diameter D2 of the clamping sleeve is adapted to the internal diameter D1 of the bore 4 of the bone-anchoring element 1 in such a way that the clamping sleeve 10 can be introduced into the bore 4. As is evident from FIG. 2, there is still sufficient space radially so that a filament 50 which is connected to a ligament, or the ligament itself, has space between the wall 14 of the clamping sleeve 10 and the surface 9 of the bore 4 of the bone-anchoring element 1 (cf. FIG. 2). As is evident from FIG. 4, the clamping sleeve 10 has profiling 15 on its outside. This profiling 15 is a preferred working example—the clamping sleeve 10 can also be smooth. Profiling advantageously provides a certain increased static friction.

As is further evident from FIGS. 1 and 2, the clamping sleeve 10 is connected to a spindle 20. This spindle 20 is led through the interior of the clamping sleeve 10. The proximal end 21 of the spindle 20 is extended in comparison with the distal end 22 of the spindle 20. In FIG. 5, the corresponding diameter D3 of the proximal end 21 is shown in comparison with the diameter D4 of the distal end. It is furthermore clear that the internal diameter D5 of the clamping sleeve 10 is smaller than the external diameter D3 of the spindle 20 but greater than the external diameter D4 of the spindle 20. Consequently, the spindle 20 can be led through the clamping sleeve 10 from the proximal end 11. Overall, both the external diameter D3 of the spindle 20 and the external diameter D2 of the clamping sleeve 10 can be smaller than the internal diameter D1 of the bone-anchoring element 1. Clamping sleeve 10 and spindle 20 are consequently introduced into the bore 4, or more precisely into the region 4b of the bore 4.

The ligament is fixed by radially expanding the clamping sleeve 10, as indicated by the arrows 13 in FIG. 4, by withdrawal of the spindle 20. By means of the movement in the distal direction, the clamping sleeve 10 is extended not only in the internal diameter D5 but also in the external diameter D2, as indicated by the arrow 34 (cf. FIG. 1 and 2). Thus, the wall 14 is pressed against the inner surface 9 of the bone-anchoring element 1 and the filament 50 is thus fixed. It is so to speak fixed between the bone-anchoring element 1 and the clamping sleeve 10 by jamming. As is clearly evident from FIGS. 1 and 2, first the proximal end 11 of the clamping sleeve 10 is pressed against the bone-anchoring element 1, more precisely against its inner surface 9. Only with continuing distal movement of the spindle 20 is the filament 50 or ligament fixed along the further regions of the clamping sleeve 10. The filament 50 and hence the ligament are consequently first fixed at the proximal end of the fixation. This has the advantage that shortenings or other length changes caused by the further fixing process are no longer possible. Even on initial contact of the wall 14 of the clamping sleeve 10 with the bone-anchoring element 1, the filament 50 and hence the ligament is fixed. Even if, for example, a filament loop or the like were to be formed in the further course, this would have no influence on length and tension of the filament 50 and hence on length and tension of the ligament. This is extremely advantageous since it is important for the success of the operation that the ligament is fixed in a predetermined length and hence with a predetermined tension. Another advantage of the apparatus according to the invention is that the fixing takes place between rigid elements. The inner surface 9 of the bone-anchoring element 1 already fixed in the bone is present on one side and the wall 14 of the clamping sleeve 10 is present on the other side. The filament 50 is consequently jammed between two rigid elements and thus fixed. In the prior art, on the other hand, fixing always takes place via the bone, cf. for example U.S. Pat. No. 6,517,579 B1.

The withdrawal of the spindle 20 for fixing the filament 50 can be effected, for example, by means of a tool in the manner of pop rivet pliers. Such pliers 30 are shown schematically and in section in FIGS. 1 and 2. These pliers have a front end 31 through which the spindle 20 is introduced. The front end 31 of the pliers 30 is supported axially on the distal end 3 of the bone-anchoring element 1 and the distal end 12 of the clamping sleeve 10. The spindle 20 is engaged by the engagement apparatus 33. On actuation of the pliers 30, the spindle 20 is moved out of the clamping sleeve 10 in the distal direction, as indicated by the arrow 34. As mentioned above, radial expansion of the clamping sleeve 10 and hence fixing of the filament 50 between bone-anchoring element 1 and clamping element 10 take place as a result. The front end 31 can additionally be provided with a filament guide (not shown) through which the filament 50 is led. This has the advantage that the filament always has sufficient play and is not unintentionally jammed between the front end 31 of the pliers 30 and the distal end 3 of the bone-anchoring element.

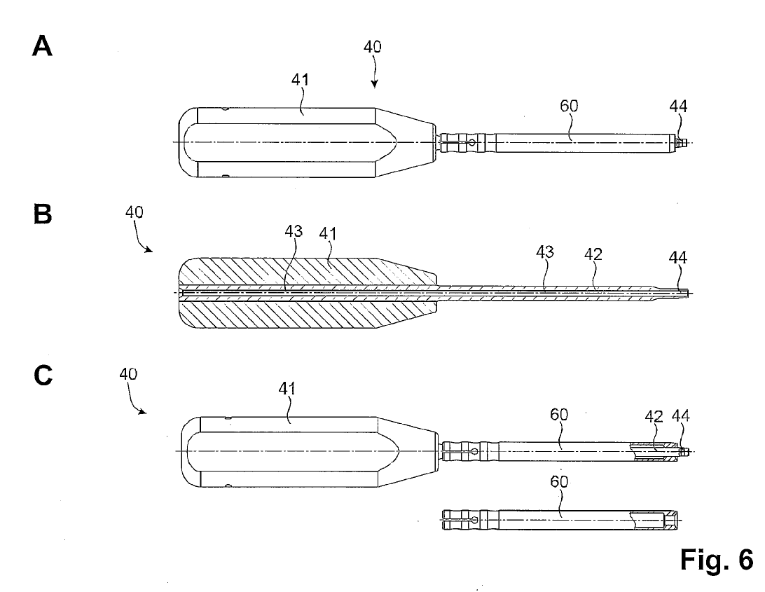

FIG. 6 shows a screwdriver 40 according to the invention in side view (A; C: partly cut-away diagram) and in section (B). It has a handle 41 and a shaft 42. The engagement region 44 is designed so that the engagement opening 7 of the bone-anchoring element 1 can be engaged in an interlocking manner therewith. The screwdriver 42 is cannulated so that the filament 50 can be led through it. The channel 43 is shown in FIG. 6B. Via the shaft 42, a guide sleeve 60 is pushed on and clamped thereon preferably by a frictional connection. The guide sleeve 60 is removable (cf. FIG. 6C). As described in more detail below, the guide sleeve 60 serves for keeping the opening through the skin and so-called soft tissues opened after removal of the screwdriver 40 after the bone-anchoring element 1 has been screwed into the bone, and as a guide during introduction of the clamping sleeve 10 connected to the spindle 20. After the clamping sleeve 10 has been introduced into the bore 4, the guide sleeve 60 is removed and the clamping sleeve 10 is radially expanded by withdrawing the spindle 20 by means of the pliers described above.

FIG. 3 shows two working examples of a bone-anchoring element 1, 1′ according to the invention. The two bone-anchoring elements are in principle of the same design. The bone-anchoring element 1′ has, at its proximal end 2′, a cylindrical guide section 16 which fits into the bone bore and has proven advantageous during introduction of the bone-anchoring element 1′ into the predrilled bone. Furthermore, the engagement opening 7′ is in the form of a hexagon socket or torx (not shown).

According to the invention, the surgeon proceeds as follows after drawing in the ligament: First, the bone-anchoring element 1 is introduced into the bore or into the drilled channel in the bone. The screwdriver 40 which engages the bone-anchoring element 1 is used for this purpose. The filament 50 is first led through the bore 4 of the bone-anchoring element 1 and is then led further through the channel 43 of the cannulated screwdriver 40 with the aid of a filament introduction aid not shown. During introduction and screwing in to the bone, the surgeon now has adequate guidance which is provided for him by the filament 50. Furthermore, the cannulated screwdriver 40 and the bore 4 of the bone-anchoring element 1 ensure that the filament 50 of the ligament is not damaged while the bone-anchoring element 1 is being screwed in and is also not jammed between screw and drilled channel. After mounting of the bone-anchoring element 1, the screwdriver 40 is withdrawn. However, the guide sleeve 60, which has been introduced together with the screwdriver 40, remains on the bone-anchoring element 1. The filament now runs in the bone-anchoring element 1 and is led to the outside through the guide sleeve 60. Thereafter, the surgeon leads the clamping sleeve 10 of the bone-anchoring element together with the spindle 20 through the guide sleeve 60 into the bone-anchoring element 1 so that the filament 50 is led correctly between the inner surface 9 of the bone-anchoring element 1 and the wall 14 of the clamping sleeve 10, as shown in FIG. 2. After the removal of the guide sleeve 60, the surgeon carries out the adjustment of the tension of the ligament, as known in the prior art, for example also with the aid of a spring balance. Once the desired tension has been set, the spindle 20 is withdrawn from the clamping sleeve 10, as described above, for example with the aid of pliers 30. After the clamping sleeve 10 has been radially extended, i.e. the spindle 20 has been withdrawn from the apparatus, the pliers 30 can be removed. The bone-anchoring element 1 is mounted and the ligament is fixed.

### LIST OF REFERENCE NUMERALS

1 Bone-anchoring element

2 Proximal end

3 Distal end

4 Bore

5 Axis

6 Thread

7 Engagement opening

8 Stop

9 Inner surface

10 Clamping sleeve

11 Proximal end

12 Distal end

13 Arrow

14 Wall

15 Profiling

16 Cylindrical guide section

20 Spindle

21 Proximal end

22 Distal end

30 Pliers

31 Front end

33 Engagement apparatus

34 Arrow

40 Screwdriver

41 Handle

42 Shaft

43 Channel

44 Engagement region

50 Filament

60 Guide sleeve

D1 Internal diameter of the bore

D2 External diameter of the clamping sleeve 10

D3 Head diameter of the spindle

D4 Shaft diameter of the spindle

D5 Internal diameter of the clamping sleeve 10

1. An apparatus for fixing a ligament, comprising a bone-anchoring element having a proximal end and a distal end, which has a bore running in a longitudinal direction, and comprising a clamping sleeve having a proximal end and a distal end, which can be accommodated in the bore and which is radially expandable, wherein the ligament can be fixed between the clamping sleeve and the inner bore of the bone-anchoring element.

2. The apparatus as claimed in claim 1, wherein the clamping sleeve is connected or can be detachably connected to a spindle, the spindle having a proximal end and a distal end, which is led through the clamping sleeve, an external diameter of the spindle being greater than an internal diameter of the clamping sleeve and the clamping sleeve being radially expandable by withdrawing the spindle from the clamping sleeve by a movement in a distal direction.

3. The apparatus as claimed in claim 1, wherein the clamping sleeve is radially expandable by screwing in a screw, in particular a grub screw, or by introducing a rivet.

4. The apparatus as claimed in claim 1, wherein the bone-anchoring element has a thread, in particular a bone thread, on its outside.

5. The apparatus as claimed in claim 1, wherein the bone-anchoring element is designed without a macrostructure, in particular without a bone thread, on its outside.

6. The apparatus as claimed in claim 1, wherein the distal end of the bone-anchoring element has a larger diameter in comparison with the proximal end.

7. The apparatus as claimed in claim 1, wherein the bone-anchoring element has at least one engagement opening at its distal end.

8. The apparatus as claimed in claim 1, wherein the bone-anchoring element is itself in the form of clamping sleeve, in particular a star-shaped clamping sleeve, which can be anchored in a bone.

9. The apparatus as claimed in claim 1, wherein the ligament is connected at each end to at least one filament which can be fixed between the clamping sleeve and the bore of the bone-anchoring element by radial expansion of the clamping sleeve, such that the ligament can be fixed.

10. The apparatus as claimed in claim 1, wherein an outside of the clamping sleeve is structured, in particular ribbed, knurled or toothed.

11. The apparatus as claimed in claim 1, wherein the bone-anchoring element has a cylindrical guide section at its proximal end.

12. The apparatus as claimed in claim 1, wherein the bone-anchoring element is produced from metal, in particular from titanium or a titanium alloy, bone material, in particular of bovine or human origin, bioresorbable materials, in particular polylactide, or a shape memory alloy.

13. The apparatus as claimed in claim 1, wherein the engagement opening of the bone-anchoring element has the shape of a groove, of a hexagon socket or of a torx.

14. The apparatus as claimed in claim 1, wherein a surface of the bone-anchoring element which is on the bone side is roughened.

15. A screwdriver for a bone-anchoring element of the apparatus as claimed in claim 1, comprising a handle and a shaft, a channel through which a filament connected to one end of the ligament can be led and can be connected to the bone-anchoring element passing at least partly through the handle and/or the shaft, and the shaft having, at its free end, an engagement region with rotary driver means for the introduction of the bone-anchoring element, wherein a guide sleeve which can be pushed onto the shaft and pulled off therefrom and which forms an axial stop for the bone-anchoring element on screwing in of the bone-anchoring element is provided.

16. A method for the surgical repair of a ligament which, after a replacement for the ligament to be repaired has been drawn in via at least one drilled channel, comprising the steps of:
a) introducing a bone-anchoring element into the drilled channel and anchoring therein, the ligament or at least one filament connected thereto for fixation being led in a bore of the bone-anchoring element, and
b) expanding radially a clamping sleeve accommodated in the inner bore, with the result that the ligament can be fixed between the clamping sleeve and the bore of the bone-anchoring element.

17. The method as claimed in claim 16, the radial expansion being effected by means of a spindle which is led through the clamping sleeve, by withdrawing the spindle, whose external diameter is greater than an internal diameter, of the clamping sleeve from the clamping sleeve.

18. The method as claimed in claim 16, wherein at least the anchoring of the bone-anchoring element in the drilled channel is effected by means of a cannulated screwdriver in whose channel the at least one filament is guided.

19. The method as claimed in claim 16, wherein the clamping sleeve and/or the bone-anchoring element is produced from a shape memory alloy and the radial expansion being effected by triggering the incorporated memory effect by heating of the memory metal.

20. A screwdriver for a bone-anchoring element of the apparatus as claimed in claim 1, comprising a handle and a shaft, a channel through which a filament connected to one end of the ligament can be led and can be connected to the bone-anchoring element passing at least partly through the handle and/or the shaft, and the shaft having, at its free end, an engagement region with rotary driver means for the introduction of the bone-anchoring element, wherein a guide sleeve which can be pushed onto the shaft and can be pulled off therefrom and which forms an axial stop for the bone-anchoring element on screwing in of the bone-anchoring element and forms a fixed working channel for the introduction and expansion of a clamping sleeve by means of a spindle after screwing in of the bone-anchoring element and removal of the shaft is provided.
